(19) United States
(12) Patent Application Publication
(10) Pub. No.: US 2024/0108565 A1
(43) Pub. Date: Apr. 4, 2024

(54) COMPOSITION COMPRISING A HYDROLYZED KERATIN AND A SILANE CROSSLINKING AGENT AND PROCESS FOR HAIR PROTECTION DURING BLEACHING

(71) Applicant: Federici Brands, LLC, Wilton, CT (US)

(72) Inventor: Joseph CINCOTTA, PhD, Trumbull, CT (US)

(21) Appl. No.: 18/274,859

(22) PCT Filed: Jan. 25, 2022
(86) PCT No.: PCT/US2022/013673

§ 371 Date: Jul. 28, 2023

Related U.S. Application Data
(60) Provisional application No. 63/144,538, filed on Feb. 2, 2021.

(51) Int. Cl. A61K 8/65; A61K 8/04; A61K 8/58; A61Q 5/08

(52) U.S. Cl. CPC A61K 8/65; A61K 8/046; A61K 8/585; A61Q 5/08; A61K 2800/95

(57) ABSTRACT

Bleach protection agents and a bleaching pre-treatment method are disclosed. The bleach protection agents comprise an aqueous mixture comprising hydrolyzed keratin and a silane crosslinking agent. Pre-treated hair is dried to deposit a protective keratin-based film on the surface of the hair. Thereafter, a bleaching composition is applied to the protected hair. Upon exposure to the bleaching composition, the keratin-based film is sacrificed, the hair is bleached, and damage to the hair from the bleaching composition is minimized or avoided. Surprisingly, bleaching of the underlying hair is effectively accomplished while also preserving its elasticity, strength, smoothness, and overall quality.

### FIELD OF THE INVENTION

The invention relates to a composition and process for protecting hair during bleaching and to pretreatment of hair to be bleached with a sacrificial keratin-based polymer film.

### BACKGROUND OF THE INVENTION

Hair bleaching or coloring is ubiquitous in developed countries, particularly among women. Regular treatments, typically every 6-8 weeks, are needed. Hair colorants and bleaches that contain hydrogen peroxide make up about 80% of the market for these products. In the colorants, hydrogen peroxide both lightens the underlying hair color and oxidizes dye precursors to generate the desired color.

High-energy species generated during bleaching (e.g., the perhydroxy anion and reactive oxygen species such as hydroxyl radicals) weaken or damage hair protein, which is predominantly keratin, by attacking disulfide crosslinks and, to a lesser extent, peptide bonds of the keratin. The processed hair becomes brittle and inelastic. Subsequent bleaching aggravates the problem because areas of the hair that were already weak from an earlier bleaching treatment are exposed again to the harsh chemicals. These small areas of overlapping treatments become susceptible to breakage. Metals found in or on the hair can accelerate further oxidative processes that damage the hair structure.

Most hair treatments seek to “repair” the seemingly inevitable damage that occurs during bleaching. For instance, formulations that include synthetic silicones or siloxane polymers can be used to impart a silkier feel to damaged hair (see, e.g., U.S. Pat. Nos. 5,679,819; 8,048,846; and 8,785,370). Bis-amino diglycol dimaleates have been included in small proportions (typically less than 0.2 wt. %) in consumer-marketed hair-care products (e.g., Olaplex® products) for repairing, strengthening, and protecting damaged hair. The related patents teach to apply the dimaleates to hair either during or after bleaching to repair damaged disulfide bonds (see, e.g., U.S. Pat. Nos. 9,326,926; 9,498,419; or 10,076,478).

Protein-siloxane polymers have been used in the context of oxidative hair coloring (see U.S. Pat. No. 10,117,825), a process that is less aggressive when compared with bleaching. According to the reference (col. 10, II. 1-12), exposure of the protein-siloxane treated hair with reagents used for oxidative coloring causes no decomposition of the protein-siloxane polymer. Thus, the polymer is not sacrificed during the coloring process.

Ways to pretreat hair prior to exposure to bleaching agents have been described. These include, for example, pretreatment with ferrous compounds or other oxidants and chelating agents (see, e.g., U.S. Pat. Nos. 3,632,295; 5,064,441; and 7,179,302), pretreatment with certain dimer fatty acids (U.S. Pat. No. 4,067,345), pretreatment with specialized silicones (U.S. Pat. No. 8,740,995), or pretreatment with a sugar, starch, or polyol (U.S. Pat. No. 5,756,077). Recent advice on the internet suggests applying coconut oil to the hair in advance of a bleaching treatment (see, e.g., https://botoxcapilar.org/en/coconut-oil-before-bleaching-hair/) to minimize damage.

Keratin- or other protein-based materials are sometimes included in consumer-based hair-care products for damage repair or other purposes (see, e.g., U.S. Pat. Nos. 6,013,250 and 8,785,370).

The industry would benefit from the availability of better ways to avoid damage to hair caused by at-home or in-salon bleaching. Known pretreatment methods have proved largely unsuccessful and have been superseded by methods and consumer products aimed at damage repair. Ideally, the hair could be effectively protected prior to exposure to bleaching agents or other harsh chemicals.

### SUMMARY OF THE INVENTION

In one aspect, the invention relates to a hair bleaching method. The method comprises first pretreating a subject's hair with a bleach protection agent. The bleach protection agent comprises an aqueous mixture comprising hydrolyzed keratin and a silane crosslinking agent. The pre-treated hair is blow dried or is allowed to dry such that a protective keratin-based film forms on the surface of the hair. Thereafter, a bleaching composition is applied to the protected hair. Upon exposure to the bleaching composition, the keratin-based film is sacrificed, the hair is bleached, and damage to the hair from the bleaching composition is minimized or avoided.

In other aspects, the invention relates to compositions useful as bleach protection agents, including aqueous spray formulations; lotions, creams, or gels; and dilutable concentrates. Each of these compositions comprises, in part, (a) hydrolyzed keratin or its mixture with keratin, and (b) a silane crosslinking agent.

Damage to hair from conventional bleaching processes can be minimized or avoided when hair is pretreated to deposit a protective, keratin-based film on the surface of the hair prior to its exposure to bleaching compositions. When the bleaching composition is applied, the protective film is sacrificed. Surprisingly, bleaching of the underlying hair is effectively accomplished while also preserving its elasticity, strength, smoothness, and overall quality.

### DETAILED DESCRIPTION OF THE INVENTION

### Hair Bleaching Method

In one aspect, the invention relates to a hair bleaching method that utilizes a bleach protection agent, which is formed in two steps. In a first step, a subject's hair is pretreated with a bleach protection agent. The bleach protection agent comprises an aqueous mixture comprising hydrolyzed keratin and a silane crosslinking agent.

The hair bleaching method, although primarily aimed at human subjects, might also be applied to non-human subjects, such as pets or other reasonably cooperative domesticated animals.

The aqueous mixture can be in the form of an aqueous solution, suspension, emulsion, microemulsion, lotion, cream, or gel. Aqueous solutions are convenient because they can be sprayed onto the hair for uniform coverage. In some cases, it may be more desirable to have a lotion, cream, or gel to apply to a smaller, more targeted area, such as hair to be bleached near the scalp. In some aspects, the bleach protection agent is applied in the form of a spray, lotion, cream, or gel on previously bleached hair, on new growth areas close to the scalp, or both, or on a portion or the entire length of hair for highlighting or full-head bleaching.

In some aspects, separate aqueous mixtures comprising hydrolyzed keratin and silane crosslinking agent can be applied contemporaneously, but in practice, it is more convenient to use an aqueous mixture that includes both components.

The subject's hair can be pretreated with the bleach protection agent by any suitable method, including applying by spraying, eye dropper application, brushing, sponging, dabbing, massaging, or the like, onto areas that will be treated subsequently with the bleaching agent.

In a second step, the subject's pre-treated hair is blow dried or is allowed to dry such that a keratin-based film forms on the surface of the hair. Blow drying is preferred because the heat activates and promotes a film-formation reaction between the silane crosslinking agent and the hydrolyzed keratin and any other reactive components in the bleach protection agent (e.g., other hydrolyzed proteins or acrylate-functional crosslinking agents). The protective film will ultimately be sacrificed when the hair is exposed to the bleaching agent. However, damage to the underlying hair will be minimized or avoided by including the pre-treatment and film-forming steps.

In a third step, a bleaching composition is applied to the protected hair. The bleaching step utilizes conventional bleaching compositions that are applied using conventional techniques. The bleaching compositions are commonly formulated by combining bleaching powder with a developer cream (typically about 1 part of bleaching powder to about 2 parts of developer) and blending until a homogeneous bleaching paste is formed. Bleaching powders commonly include persulfate and/or percarbonate salts, thickeners, silica, conditioners, and other components. Developers contain various proportions of hydrogen peroxide. Combination of the bleaching powder and developer generates various anionic and high-energy, free-radical species that decompose or sacrifice the protective film and bleach the hair while avoiding or minimizing damage to the underlying hair from reactive oxygen species. Typical bleaching compositions are described, for example, in U.S. Pat. Nos. 5,575,989 and 8,623,339.

Removal of residues from the bleach protection agent is straightforward. Typically, residue from the sacrificed keratin-based film is removed from the bleached hair by rinsing, shampooing, conditioning, or combinations thereof.

### Bleach Protection Agent

In some aspects, the invention relates to a bleach protection agent. The bleach protection agent comprises a mixture of hydrolyzed keratin (or its combination with keratin) and a silane crosslinking agent. The bleach protection may include other components, such as chelating agents, preservatives, solvents, pH adjusting agents, surfactants, emulsifiers, emollients, rheology modifiers, and hydrolyzed proteins other than hydrolyzed keratin. When pre-treated hair is blow-dried, the added heat promotes a film-forming reaction between the hydrolyzed keratin and the silane crosslinking agent.

Hydrolyzed Keratin

The aqueous mixture comprises hydrolyzed keratin and may also include (unhydrolyzed) keratin. (As indicated further below under “Optional components,” hydrolyzed vegetable proteins, hydrolyzed grain proteins, and other similar products can also be included). The degree of hydrolysis of the keratin is not critical. Suitable commercial products are commonly mixtures of hydrolyzed keratin and other components, especially keratin and/or hydrolyzed vegetable proteins. Commercial products include, e.g., Croda products: KERATEC™ IFP PE (hydrolyzed keratin/keratin mixture), KEREFFECT™ SD (hydrolyzed keratin), PROSINA™ (high-cystine hydrolyzed keratin), CROTEIN™ K (hydrolyzed keratin), CROTEIN™ WKP (hydrolyzed keratin), KERASOL™ (hydrolyzed keratin), and CROTEIN™ Cashmere PE (hydrolyzed keratin); BASF products: NUTRILAN® KERATIN LM (hydrolyzed keratin), NUTRILAN® KERATIN W (hydrolyzed keratin), NUTRILAN® KERATIN W PP (hydrolyzed keratin), and CASHMILAN® LS 9604 (hydrolyzed keratin); Lonza's HYDROKERATIN AL-30 (hydrolyzed keratin); Seiwa Kasei products: PROMOIS™ WK series (hydrolyzed keratin) and PROMOIS™ KR-30 (hydrolyzed keratin); Tri-K's KERA-TEIN™ V NPNF, and hydrolyzed keratins available from other suppliers.

Suitable hydrolyzed keratins have number-average molecular weights, as measured by one or more of gel-permeation chromatography, SDS-polyacrylamide gel electrophoresis, size-exclusion high-performance liquid chromatography, wet-chemical analysis (e.g., hydroxyl number measurement), mass spectrometry analysis, or other analytical techniques within the range of 500 g/mol to 500,000 g/mol, or from 500 g/mol to 50,000 g/mol, or from 500 to 5,000 g/mol, or from 500 to 1,500 g/mol. The molecular weight distribution can be uni-, bi-, or multimodal. For example, KERATEC™ IFP PE has a bimodal molecular weight distribution with components having molecular weights ranging from both 40,000 to 60,000 g/mol and 3,000 to 4,000 g/mol.

The amount of hydrolyzed keratin (or its combination with keratin) used depends on the type of formulation. Aqueous mixtures may contain as little as 3 wt. % of the hydrolyzed keratin based on the amount of aqueous mixture; dilutable concentrates may contain as much as 70 wt. % of the hydrolyzed keratin.

### Silane Crosslinking Agent

The silane crosslinking agent can react with the hydrolyzed keratin to form a continuous, protective film on the surface of hair when a mixture of the crosslinking agent and hydrolyzed keratin is applied to the hair and heated (or allowed to dry) to remove moisture. Suitable silane crosslinking agents include at least one silicon atom that is bonded to one or more, typically two or three, easily hydrolyzed atoms or groups. Examples include silyl halides, silanols, and alkoxysilanes. Any of these can form siloxane (Si—O—Si) links by hydrolysis and condensation reactions. The silyl halide, silanol, or alkoxysilane functionalities also react readily with terminal or side-chain amino groups of hydrolyzed proteins, including hydrolyzed keratins. The silane crosslinking agents frequently include other functional groups that react with amino groups, such as epoxides, anhydrides, acid halides, sulfonyl halides, and the like. The silane crosslinking agent can, and frequently does, incorporate a hydrolyzed protein in its structure provided it retains a silyl halide, silanol, alkoxysilane, or other easily hydrolyzed silicon-containing functional group. In some cases, the silane crosslinking agent incorporates polyether moieties, quaternized amines, non-reactive amines, and/or other functional groups in addition to one or more silane crosslinking functionalities.

Suitable silane crosslinking agents for use herein and their methods of preparation have been previously described. See, e.g., U.S. Pat. Nos. 5,412,074; 5,679,819; 8,048,846; and 10,117,825, the teachings of which are incorporated herein by reference. In some aspects, the silane crosslinking agents include cystine bis-PG-propyl silanetriol, hydrolyzed vegetable protein PG-propyl silanetriol, or a combination thereof.

Suitable silane crosslinking agents are also commercially available. Examples include CRODASONE™ cystine PE (cystine bis-PG-propyl silanetriol), CRODASONE™ W (hydrolyzed wheat protein PG-propyl silanetriol), CRODASONE™ P (hydrolyzed pea protein PG-propyl silanetriol), and KERAVIS™ PE (hydrolyzed vegetable protein PG-propyl silanetriol), products of Croda; SILQUEST™ A-187 silane and SILSOFT™ CLX-E conditioning agent (products of Momentive); DYNASYLAN® GLYEO (3-glycidyloxypropyltriethoxysilane) and DYNASYLAN® GLYMO (3-glycidyloxypropyl-trimethoxysilane), products of Evonik; PROMOIS™ WK-HSIGF (hydrolyzed keratin PG-propyl methylsilanediol), PROMOIS™ WS-HSIG (hydrolyzed soy protein PG-propyl methylsilanediol), PROMOIS™ WG-SIG (hydrolyzed wheat protein PG-propyl methylsilanediol), PROMOIS™ GOMA-SIG (hydrolyzed sesame protein PG-propyl methylsilanediol), PROMOIS™ S-700SIGF (hydrolyzed silk PG-propyl methylsilanediol), PROMOIS™ WU-32SIG (hydrolyzed collagen PG-propyl methylsilanediol), products of Seiwa Kasei; and similar epoxy-functional silanes.

The amount of silane crosslinking agent used depends on the type of formulation. Aqueous mixtures may contain as little as 6 wt. % of the silane crosslinking agent based on the amount of aqueous mixture; dilutable concentrates may contain as much as 90 wt. % of the silane crosslinking agent.

Previously, similar silane crosslinking agents have been described as minor (or trace) components of hair conditioning formulations. Siloxane polymers have long been known to impart a silky feel when used to condition freshly shampooed hair. Certain proteins are also considered valuable for improving gloss, combability, body, and moisture control in hair. Consequently, silane crosslinking agents that incorporate proteins were developed to take advantage of the attributes of both siloxane polymers and proteins (see, e.g., U.S. Pat. Nos. 5,412,074 and 8,048,846). However, the same polymers have not previously been suggested for use at a much higher proportion to form a protective film on the hair as a bleaching pretreatment step.

Other Components

The bleach protection agent can comprise other components, including, for example, chelating agents, preservatives, solvents, pH adjusting agents, surfactants, emulsifiers, emollients, rheology modifiers, and hydrolyzed proteins other than hydrolyzed keratin.

Aside from hydrolyzed keratin, hydrolyzed proteins from animal, vegetable, or fermentation sources can be included as optional components of the bleach protection agents. For instance, hydrolyzed proteins made by alkaline, acidic, or enzymatic hydrolysis of wheat, potato, rice, soy, silk, milk, cottonseed, collagen, elastin, casein, or other protein sources can be included. The hydrolyzed proteins can be chemically modified, e.g., by quaternizing a portion of the available amino groups or by reaction with acrylate monomers to introduce acrylic functionality. For examples of hydrolyzed proteins and their chemically modified varieties, see U.S. Pat. Nos. 8,048,846; 10,117,825; and 10,695,272, the teachings of which are incorporated herein by reference.

The bleach protection agent can include a small proportion, typically 0.01 to 1 wt. %, or 0.05 to 0.2 wt. %, or about 0.1 wt. %, based on the amount of formulated bleach protection agent, of one or more chelating agents that sequester metal ions. Examples include ethylenediamine tetraacetic acid (EDTA) salts such as disodium EDTA, dipotassium EDTA, tetrasodium EDTA, and the like.

The bleach protection agent can include a minor proportion of one or more preservatives. Phenoxyethanol, or its combination with a glycol diluent, especially caprylyl glycol, is suitable for use and effective as a biocide. Other suitable preservatives include 1,2-hexanediol, benzyl alcohol, sodium benzoate, potassium sorbate, methylisothiazolinone, and their mixtures, and the like. The preservative is typically used in an amount within the range of 0.3 to 2 wt. %, or 0.75 to 1.5 wt. %, based on the amount of formulated bleach protection agent. OPTIPHEN™ preservatives, products of Ashland (e.g., OPTIPHEN™ 200, OPTIPHEN™ 300, and OPTIPHEN™ PO), are commercially available examples.

The bleach protection agent can include a minor proportion, typically 0.5 to 3 wt. % based on the amount of formulated bleach protection agent, of one or more organic solvents. Examples include 1,3-propanediol, propylene glycol, dipropylene glycol, ethanol, glycerin, and combinations thereof.

The bleach protection agent can include a pH adjusting agent, typically citric acid, in an amount effective to adjust the pH to a value within the range of 7.0 to 9.0, or from 7.5 to 9.0, or especially from 8.0 to 8.5.

The bleach protection agent can include other components such as surfactants, emulsifiers, emollients, rheology modifiers. The proportion of these traditional components of personal care products is generally greater in lotions, creams, gels, and dilutable concentrates when compared with aqueous spray formulations, which may have little or none of these components.

In some aspects, the bleach protection agent is formulated as an aqueous solution or microemulsion intended for spray application. In other aspects, the bleach protection agent is formulated as an oil-in-water or water-in-oil emulsion to form a lotion, cream, or gel that can be applied in a more targeted way when compared with a spray. In still other aspects, the bleach protection agent can be formulated as a water-dilutable concentrate.

The bleach protection agent can (and usually does) include water in an amount appropriate for the type of formulation desired. A dilutable concentrate may include little or no water, while a cream, lotion, or gel will contain some water, and a spray formulation will contain water as a principal component. For instance, a spray formulation might contain 70 to 95 wt. % of water; a lotion might contain 20 to 70 wt. % of water; a cream might contain 10 to 30 wt. % water; a gel formulation might contain 1 to 10 wt. % of water; and a dilutable concentrate might contain 0 to 5 wt. % of water.

The bleach protection agent can be an aqueous spray formulation. In some aspects, these compositions comprise (a) 70 to 95 wt. % of water; (b) 3 to 10 wt. % of hydrolyzed keratin or its mixture with keratin; and (c) 6 to 18 wt. % of a silane crosslinking agent, wherein the wt. % amounts are based on the amount of aqueous spray formulation. In some aspects, the pH of the aqueous spray formulation is adjusted to a pH within the range of 7.0 to 9.0, or from 8.0 to 8.5. In some aspects, the aqueous spray formulation comprises 3 to 7 wt. % of hydrolyzed keratin or its mixture with keratin, and 7 to 14 wt. % of the silane crosslinking agent. In other aspects, the aqueous spray formulation further comprises 0.5 to 3.0 wt. % of 1,3-propanediol, glycerin, propylene glycol, dipropylene glycol, ethanol, or mixtures thereof.

The bleach protection agent can be formulated as a lotion, cream, or gel. A lotion can be mostly water, while a gel can be formulated with very little water. Thus, in some aspects, the bleach protection agent formulated as a lotion, cream, or gel comprises (a) 1 to 70 wt. % of water; (b) 3 to 20 wt. % of hydrolyzed keratin or its mixture with keratin; and (c) 6 to 40 wt. % of a silane crosslinking agent. The wt. % amounts are based on the amount of the lotion, cream, or gel. In some aspects, the pH of the lotion, cream, or gel is adjusted to a pH within the range of 7.0 to 9.0, or from 8.0 to 8.5.

The bleach protection agent can be formulated as a water-dilutable concentrate. The concentrates comprise (a) 10 to 70 wt. %, 15 to 60 wt. %, or 20 to 45 wt. % of hydrolyzed keratin or its mixture with keratin; and (b) 30 to 90 wt. %, 40 to 85 wt. %, or 55 to 80 wt. % of a silane crosslinking agent. The wt. % amounts are based on the combined amounts of (a) and (b). Formulating the bleach protection agent as a water-dilutable concentrate may be effective for reducing packaging and shipping costs. In these aspects, it may be most desirable to supply the bleach protection agent as a two-part kit, with components (a) and (b) to be combined, optionally with the other components described herein, and diluted by the hair care professional as needed.

As indicated above, the aqueous spray formulations, lotions, creams, gels, or dilutable concentrates can further comprise at least one component selected from the group consisting of chelating agents, preservatives, solvents, pH adjusting agents, and hydrolyzed proteins other than hydrolyzed keratin.

### Analysis of Hair Samples

Amino acid analysis of samples of bleached hair that are either untreated or treated with the inventive bleach protection agents reveals the degree of protection available from the pre-treatment. In some aspects, amino acid analysis of the bleached hair following removal of the bleaching composition and any residue from the protective keratin-based film demonstrates that the bleached hair has at least 10%, or at least 20%, or at least 30%, typically 10% to 30%, more cystine content when compared with a hair sample bleached the same way in the absence of the keratin-based film.

In other aspects, amino acid analysis of the bleached hair following removal of the bleaching composition and any residue from the protective keratin-based film demonstrates that the bleached hair has at least 5%, at least 10%, or from 10% to 50% more of each proteinogenic amino acid other than cysteine when compared with a hair sample bleached the same way in the absence of the keratin-based film (see Table 3, last column). The proteinogenic amino acids are those listed in Table 3, except for ornithine, which is a non-proteinogenic amino acid. These results suggest that the level of damage is reduced considerably when, prior to bleaching, the virgin hair is pretreated with an aqueous mixture comprising hydrolyzed keratin and a silane crosslinking agent and then dried to form a protective film according to the invention. Minor damage to primary structure is still apparent, but the difference when compared with bleach treatment of unprotected hair is remarkable.

The following examples are only illustrations; the claims define the scope of the inventive subject matter.

### Bleach Protection Spray Compositions

Aqueous protection sprays are made by combining the components listed in Table 1 and mixing until a homogeneous solution is obtained.

TABLE 1
Bleach protection spray compositions

| | Range | | | |
|---|---|---|---|---|
| Component | (wt. %) | Ex. 1 | Ex. 2 | Ex. 3 |
| KERATEC ™ IFP PE (mixture | 3.0 to 10.0 | 7.0 | 4.0 | 3.0 |
| of keratin and hydrolyzed | | | | |
| keratin) | | | | |
| CRODASONE ™ Cystine PE | 6.0 to 18.0 | 7.0 | 7.0 | 11.0 |
| (cystine bis-PG-propyl | (combined) | | | |
| silanetriol) | | | | |
| KERAVIS ™ PE (hydrolyzed | | 0 | 3.0 | 0 |
| vegetable protein PG-propyl | | | | |
| silanetriol) | | | | |
| disodium EDTA | 0.05 to 0.20 | 0.10 | 0.10 | 0.10 |
| 1,3-propanediol | 1.0 to 2.0 | 2.0 | 2.0 | 1.0 |
| OPTIPHEN ™ (mixture | 1.0 to 1.2 | 1.1 | 1.1 | 1.1 |
| of phenoxyethanol and | | | | |
| caprylyl glycol) | | | | |
| citric acid | 0.05 to 0.20 | 0.13 | 0.07 | 0.17 |
| purified water | q.s. to 100 | 82.67 | 82.73 | 83.63 |
| pH | 7.5 to 8.5 | 7.6 | 8.0 | 8.15 |

### Pretreatment of Hair Swatches

Dry, pre-swatched samples of virgin dark blonde hair (3.0 g, 6″ length, supplied by International Hair Importers, NY) are saturated with a bleach protection spray composition as shown in Table 1, and the samples are blow dried at 60° C. for 2 min. A noticeable film is deposited on the hair.

### Bleaching

Each swatch is placed on a sheet of aluminum foil. Bleach paste (prepared by mixing until smooth a mixture of Wella Powder Lightener (1 part by weight, product of Color Charm) and 30 Vol Crème Developer (product of Salon Care)) is applied with a flat coloring brush to both sides of the swatch. The swatch is wrapped in aluminum foil and placed in an oven at 45° C. for 20 min. The swatch is then removed from the foil, rinsed for 2 min. with warm (37° C.) water, washed twice with a sulfate-free shampoo, and thoroughly rinsed again for 0.5 min. The swatch is blow dried and allowed to stand at room temperature for 24 h. Thereafter, the entire process is repeated on the already-bleached swatch.

In control experiments, swatches from the same lot of hair are bleached as described above except that the pretreatment step with the bleach protection spray composition is omitted.

### Analysis of Treated Samples

An outside testing laboratory (Biosynthesis, Lewisville, TX) performs amino acid analysis on the bleached hair samples described above according to the following procedure. A bleached hair sample (5.0 mg) is combined in a hydrolysis tube with a mixture of 1 wt. % phenol in 6 N hydrochloric acid (200 μL). The mixture is heated at 110° C. for 24 h, then dried. The resulting extracted amino acids are reconstituted in ultra-pure water (20 mL). A 50-μL aliquot of the solution is used for analysis of amino acid content. Results appear in Table 2.

TABLE 2
Amino Acid Analysis of Treated Hair Swatches (μg/mg)

| | Control | Test | Test | Control | Test | Control | Test |
|---|---|---|---|---|---|---|---|
| | A | A1 | A2 | B | B | C | C |
| spray | none | Ex. 1 | Ex. 1 | none | Ex. 2 | none | Ex. 3 |
| composition | | | | | | | |
| cysteic acid | 64.5 | 67.7 | 61.2 | 87.8 | 71.4 | 74.8 | 70.9 |
| cysteine | 2.68 | 1.96 | 3.51 | 8.41 | 10.5 | 9.39 | 10.2 |
| cystine | 60.1 | 74.3 | 74.2 | 63.4 | 79.2 | 78.7 | 88.1 |
| % increase | | 23.4 | 23.4 | | 25.0 | | 11.9 |
| in cystine | | | | | | | |
| v. control | | | | | | | |

As shown in Table 2, the analyzed hair swatch samples following bleaching treatment have a substantially higher cystine content if the swatch is pretreated with an inventive bleach protection spray in advance of the usual bleaching step.

When a complete analysis of all amino acids is obtained (see Table 3, reporting all results from Control A and Test A1, above, which are representative), it is apparent that bleaching disturbs not only the disulfide crosslinks but also damages the primary structure of a sizable portion of the keratin protein that makes up about 85+% of the composition of hair as well as other hair proteins. When virgin, unprotected hair is bleached, the level of almost all of the proteinogenic amino acids that make up the keratin protein is reduced. The only exception is cysteic acid, which increases when disulfide bonds in cystine are destroyed or sulfhydryl bonds in cysteine are oxidized. (Ornithine is a non-proteinogenic amino acid.) The level of proteinogenic amino acids in the protected, bleach-treated hair samples is at least 5%, or at least 10%, or from 10% to 50% greater than that found in the unprotected, bleach-treated hair samples (see Table 3, last column). The damaged keratin is lost when the bleached hair is washed and/or rinsed. In contrast, the level of damage is reduced considerably when, prior to bleaching, the virgin hair is pretreated with an aqueous mixture comprising hydrolyzed keratin and a silane crosslinking agent and then dried to form a protective film according to the invention. Minor damage to primary structure is still apparent, but the difference when compared with bleach treatment of unprotected hair is remarkable.

### Salon Evaluation of Bleach Protection Spray

Professional colorists, two from the United States and one from the United Kingdom, are asked to evaluate the performance of the inventive bleach protection spray on each of three panelists. The panelists have hair colors ranging from dark blonde to medium brown. Each panelist arrives at the salon with dry hair, which is parted in the middle. The hair on one side of the part is saturated with the inventive protection spray, then rough dried with a warm blow dryer. Bleach mixture is prepared using a 2:1 (w/w) ratio of 30 Volume developer and bleach powder lightener. The bleach mixture is applied to both sides of the head and processes in foil or in air at room temperature for 20-30 minutes. The hair is rinsed with warm water for 3 minutes, then shampooed twice, conditioned, and rinsed again. Colorists are asked to evaluate wet elasticity, wet strength, dry smoothness, lightening (dry), and overall condition of the hair using the following scale: 5=superior to control; 4=better than control; 3=equal to control; 2=slightly worse than control; 1=worse than control. Results of the evaluation appear in Table 4.

As shown in Table 4, all of the colorists considered the pretreated hair for all of the panelists to be superior or better than the control in wet elasticity, wet strength, dry smoothness, and overall condition. For Panelist #2, two of the three colorists noticed that the lightening effect of the bleach was slightly diminished by the presence of the protective coating when compared with the control (unprotected) side, but for Panelists #1 and #3, the experts considered the lightening effect to be equal. Overall, the results suggest that pretreatment according to the invention gives well-bleached hair of improved elasticity, strength, smoothness, and overall quality.

TABLE 3
Complete Amino Acid Analysis of Hair Samples

| | | Virgin | Protected | | | |
|---|---|---|---|---|---|---|
| | Virgin | hair | hair | % Δ | % Δ | % Δ |
| | hair | bleached | bleached | bleached | protected | protected |
| Amino acid | (μg/mg) | (μg/mg) | (μg/mg) | v. virgin | v. virgin | v. bleached |
| cysteic acid | 9.5 | 64.5 | 67.7 | +578.6 | +612.3 | +5.0 |
| aspartic acid | 45.7 | 41.1 | 45.9 | −10.1 | +0.2 | +11.5 |
| threonine | 53.0 | 46.5 | 52.4 | −12.4 | −1.2 | +12.7 |
| serine | 66.7 | 55.8 | 63.3 | −16.3 | −5.1 | +13.4 |
| glutamic acid | 112.7 | 98.4 | 111.1 | −12.7 | −1.4 | +12.9 |
| cysteine | 7.5 | 2.7 | 2.0 | −64.4 | −74.0 | −26.9 |
| proline | 49.9 | 42.6 | 49.2 | −14.7 | −1.4 | +15.6 |
| glycine | 23.3 | 19.7 | 22.4 | −15.2 | −3.5 | +13.7 |
| alanine | 22.3 | 19.4 | 21.9 | −12.9 | −1.5 | +13.1 |
| valine | 39.5 | 33.4 | 38.2 | −15.5 | −3.2 | +14.6 |
| methionine | 1.9 | 1.0 | 1.5 | −47.7 | −21.5 | +50.0 |
| cystine | 101.8 | 60.1 | 74.2 | −41.0 | −27.1 | +23.5 |
| isoleucine | 22.5 | 20.0 | 22.6 | −10.8 | +0.7 | +12.9 |
| leucine | 50.8 | 44.8 | 50.3 | −11.8 | −1.0 | +12.3 |
| tyrosine | 24.6 | 17.8 | 21.4 | −27.6 | −13.0 | +20.2 |
| phenylalanine | 21.1 | 18.7 | 20.8 | −11.6 | −1.4 | +11.5 |
| ornithine | 1.2 | 1.1 | 1.1 | −6.1 | −7.8 | −1.9 |
| lysine | 19.8 | 17.2 | 19.5 | −13.2 | −1.8 | +13.2 |
| histidine | 9.7 | 6.8 | 7.9 | −30.6 | −19.4 | +16.1 |
| arginine | 71.5 | 61.7 | 69.9 | −13.8 | −2.4 | +13.3 |

TABLE 4
Professional Colorist Evaluation

| | | | | | Overall |
|---|---|---|---|---|---|
| | Elasticity | Strength | Smoothness | Lightening | condition |
| | (wet) | (wet) | (dry) | (dry) | (dry) |
| Colorist 1 | | | | | |
| (UK) | | | | | |
| Panelist 1 | 5 | 4 | 4 | 3 | 4 |
| Panelist 2 | 5 | 5 | 4 | 2 | 4 |
| Panelist 3 | 4 | 4 | 4 | 3 | 5 |
| Colorist 2 | | | | | |
| (US) | | | | | |
| Panelist 1 | 5 | 4 | 4 | 3 | 4 |
| Panelist 2 | 4 | 4 | 4 | 3 | 4 |
| Panelist 3 | 5 | 4 | 4 | 3 | 5 |
| Colorist 3 | | | | | |
| (US) | | | | | |
| Panelist 1 | 5 | 5 | 4 | 3 | 4 |
| Panelist 2 | 5 | 4 | 4 | 2 | 4 |
| Panelist 3 | 5 | 4 | 4 | 3 | 5 |

Ratings key: 5: superior to control; 4: better than control; 3: equal to control; 2: slightly worse than control; 1: worse than control.

I claim:

1. A hair bleaching method which comprises:
(a) pretreating a subject's hair with a bleach protection agent comprising an aqueous mixture comprising hydrolyzed keratin and a silane crosslinking agent;
(b) blow drying the pre-treated hair from step (a) or allowing it to dry such that a protective keratin-based film forms on the surface of the hair; and
(c) applying a bleaching composition to the protected hair from step (b);
whereby the keratin-based film is sacrificed upon exposure to the bleaching composition, the hair is bleached, and damage to the hair from the bleaching composition is minimized or avoided.

2. The method of claim 1 wherein the aqueous mixture further comprises keratin.

3. The method of claim 1 wherein the aqueous mixture further comprises at least one component selected from the group consisting of chelating agents, preservatives, solvents, pH adjusting agents, and hydrolyzed proteins other than hydrolyzed keratin.

4. The method of claim 1 wherein the silane crosslinking agent is cystine bis-PG-propyl silanetriol, hydrolyzed vegetable protein PG-propyl silanetriol, or a combination thereof.

5. The method of claim 1 wherein the bleach protection agent is applied in the form of a spray, lotion, cream, or gel on previously bleached hair, on new growth areas close to the scalp, or both, or on a portion or the entire length of hair for highlighting or full-head bleaching.

6. The method of claim 1 wherein residue from the sacrificed keratin-based film is removed from the bleached hair by rinsing, shampooing, conditioning, or combinations thereof.

7. The method of claim 1 wherein amino acid analysis of the bleached hair following removal of the bleaching composition and any residue from the protective keratin-based film demonstrates that the bleached hair has at least 10% more cystine content when compared with a hair sample bleached the same way in the absence of the keratin-based film.

8. The method of claim 1 wherein amino acid analysis of the bleached hair following removal of the bleaching composition and any residue from the protective keratin-based film demonstrates that the bleached hair has at least 20% more cystine content when compared with a hair sample bleached the same way in the absence of the keratin-based film.

9. The method of claim 1 wherein amino acid analysis of the bleached hair following removal of the bleaching composition and any residue from the protective keratin-based film demonstrates that the bleached hair has at least 10% more of each proteinogenic amino acid other than cysteine when compared with a hair sample bleached the same way in the absence of the keratin-based film.

10. A composition comprising an aqueous spray formulation, suitable for use as a bleach protection agent, the composition comprising:
(a) 70 to 95 wt. % of water;
(b) 3 to 10 wt. % of hydrolyzed keratin or its mixture with keratin; and
(c) 6 to 18 wt. % of a silane crosslinking agent;
said wt. % amounts based on the amount of aqueous spray formulation.

11. The composition of claim 10 further comprising at least one component selected from the group consisting of chelating agents, preservatives, solvents, pH adjusting agents, and hydrolyzed proteins other than hydrolyzed keratin.

12. The composition of claim 10 wherein the silane crosslinking agent is cystine bis-PG-propyl silanetriol, hydrolyzed vegetable protein PG-propyl silanetriol, or a combination thereof.

13. The composition of claim 10 having an adjusted pH within the range of 7.0 to 9.0.

14. The composition of claim 10 having an adjusted pH within the range of 8.0 to 8.5.

15. The composition of claim 10 comprising 3 to 7 wt. % of hydrolyzed keratin or its mixture with keratin, and 7 to 14 wt. % of the silane crosslinking agent.

16. The composition of claim 10 further comprising 0.5 to 3.0 wt. % of 1,3-propanediol, glycerin, propylene glycol, dipropylene glycol, ethanol, or mixtures thereof.

17. A composition comprising a lotion, cream, or gel suitable for use as a bleach protection agent, the composition comprising:
(a) 1 to 70 wt. % of water;
(b) 3 to 20 wt. % of hydrolyzed keratin or its mixture with keratin; and
(c) 6 to 40 wt. % of a silane crosslinking agent;
said wt. % amounts based on the amount of the lotion, cream, or gel.

18. The composition of claim 17 further comprising at least one component selected from the group consisting of surfactants, emulsifiers, emollients, rheology modifiers, chelating agents, preservatives, solvents, pH adjusting agents, and hydrolyzed proteins other than hydrolyzed keratin.

19. The composition of claim 17 wherein the silane crosslinking agent is cystine bis-PG-propyl silanetriol, hydrolyzed vegetable protein PG-propyl silanetriol, or a combination thereof.

20. The composition of claim 17 having an adjusted pH within the range of 7.0 to 9.0.

21. A dilutable concentrate, useful upon dilution with water as a bleach protection agent, comprising:
(a) 10 to 70 wt. % of hydrolyzed keratin or its mixture with keratin; and
(b) 30 to 90 wt. % of a silane crosslinking agent;
said wt. % amounts based on the combined amounts of (a) and (b).

22. The dilutable concentrate of claim 21 further comprising at least one component selected from the group consisting of chelating agents, preservatives, solvents, pH adjusting agents, and hydrolyzed proteins other than hydrolyzed keratin.
